(19) United States
(12) Patent Application Publication
(10) Pub. No.: US 2004/0194610 A1
(43) Pub. Date: Oct. 7, 2004

(54) Vocal pitch-training device

(76) Inventor: Monte Davis, Roswell, GA (US)

Correspondence Address: MYERS & KAPLAN, INTELLECTUAL PROPERTY LAW, L.L.C., 1899 POWERS FERRY ROAD, SUITE 310, ATLANTA, GA, 30339, US

(21) Appl. No.: 10/804,454

(22) Filed: Mar. 19, 2004

Related U.S. Application Data
(60) Provisional application No. 60/456,671, filed on Mar. 21, 2003.

(51) Int. Cl. A61F 5/58; G09B 15/06

(52) U.S. Cl. 84/466

(57) ABSTRACT

A vocal pitch-training device that provides for the holistic integration of instantaneous auditory, visual and physical biofeedback response that involve recognition of an auditory target pitch, the attempted production of a matching vocalized pitch, sensing any discordant biofeedback between the auditory target pitch and the user's vocalized pitch via the visual, auditory, and tactile biofeedback sources, and adjusting the user's vocalized pitch to match the target pitch by minimizing the discordant biofeedback as accomplished by raising or lowering the frequency of the user's pitch until a seemingly corresponding diminishment in sensation of physical biofeedback is achieved.

### CROSS-REFERENCE AND PRIORITY CLAIM TO RELATED APPLICATIONS

To the fullest extent permitted by law, the present nonprovisional patent application claims priority to and the full benefit of provisional patent application entitled “VOCAL PITCH-TRAINING DEVICE”, filed on Mar. 21, 2003, having assigned Serial No. 60/456,671.

### TECHNICAL FIELD

The present invention relates generally to vocal training devices, and more specifically to a vocal pitch-training device. The present invention is particularly suitable for, although not strictly limited to, use by amateur or professional singers for training of the singer's mind to harmonize the function of the singer's vocal chords, mouth, throat, ears and diaphragm to reproduce and sustain a specified pitch.

### BACKGROUND OF THE INVENTION

For many, vocal training plays a significant role in the development of career, personal confidence, education, and for some, in overcoming certain speech impediments.

For instance, for most orators and actors, development of proper vocal inflections could prove essential in effectively and credibly conveying a specific emotion, or the thrust of an argument. Additionally, some actors often undergo grueling language lessons to learn new languages or accents for a specific character role in a film or other performance. As such, many such professional orators and actors will spend an abundance of time in perfecting and reproducing desired inflections in their voice for effective conveyance of a speech or performance dialogue, respectively.

Further, some individuals are, unfortunately, inflicted with mental or physical disabilities that either impair normal cognitive vocal skills, or impart motor constraints thereon. Therefore, many such individuals undergo long, arduous, and often frustrating, vocal rehabilitative sessions in attempts to assist in the development, training, or re-training of their vocal skills.

With respect to amateur and professional singers, the prospect of developing a successful and sustained singing career depends upon several factors, including, personal singing style, dedication to practice regimens, and most importantly, the singer's ability to physically and mentally train himself to perfect, duplicate and sustain desired key notes or pitches (i.e., training of the vocal cords and mind, respectively).

Accordingly, for purposes of developing the requisite physical and mental capabilities for reproduction of sustained note or pitch by singers, or for developing vocal skills, in general, for any of the above-described purposes, vocal trainees, during the training session, will often receive auditory and/or visual biofeedback, generally via headphones and/or a visual LED display, respectively, that compare the trainee's generated pitch or frequency, and compare it to the target pitch or frequency; thus, enabling the singer or vocal trainee to adjust and match his/her vocal pitch or vocal inflections accordingly.

Examples of such dual-biofeedback vocal training systems may be seen with reference to U.S. Pat. No. 5,794,203 to Kehoe, and U.S. Pat. No. 4,692,117 to Goodwin. Kehoe '203 discloses a biofeedback system for speech disorders, wherein the device incorporates both auditory and visual biofeedback mechanisms for assisting an individual reach a target pitch or note via the comparison of user-produced notes or pitches with target notes or pitches, and displaying the results of the comparison via auditory and visual biofeedback; thus, permitting the user of the device to more accurately change his/her voice pitch to match the auditory and visual signals and/or pitch of the target note.

Goodwin '117 discloses an acoustic energy real-time spectrum analyzer that also utilizes auditory and visual biofeedback technology in a substantially similar fashion to assist or train a singer to develop the singer's formant and/or effectively produce the proper or desired vocal pitch.

Although both the Kehoe '203 and Goodwin '117 devices utilize auditory and visual biofeedback technology for vocal-pitch training purposes, it has become increasingly apparent that improving one's singing skills, or one's vocal skills in general, via vocal training, requires much more than the employment of visual and auditory biofeedback alone. More specifically, although such dual-biofeedback vocal training systems are effective in assisting a trainee develop his/her specific requisite vocal goals, any recognizable vocal improvement is often achieved only after devoting or expending a significant amount of training or practicing time; that is, the results take far too long to achieve for the typical eager trainee looking for substantially immediate results, or at least some indication that headway has been made. As such, many trainees utilizing present vocal training systems often become frustrated and discontinue use of same in the absence of desired results within a desired period of time.

Therefore, it is readily apparent that there is a need for a vocal pitch-training device that provides expeditious results via auditory and visual biofeedback mechanisms in addition to tactile or physical biofeedback mechanisms for a triple-biofeedback vocal pitch-training system that effectively harmonizes the function of the vocal trainee's vocal chords, mouth, throat, ears and diaphragm to permit the trainee to reproduce and sustain a specified pitch.

### BRIEF SUMMARY OF THE INVENTION

Briefly described, in a preferred embodiment, the present invention overcomes the above-mentioned disadvantages and meets the recognized need for such a device by providing a vocal pitch-training device that provides for the holistic integration of instantaneous auditory, visual and physical biofeedback response that involve recognition of an auditory target pitch, the attempted production of a matching vocalized pitch, sensing any discordant biofeedback between the auditory target pitch and the user's vocalized pitch via the visual, auditory, and tactile biofeedback sources, and adjusting the user's vocalized pitch to match the target pitch by minimizing the discordant biofeedback as accomplished by raising or lowering the frequency of the user's pitch until a seemingly corresponding diminishment in sensation of physical biofeedback is achieved.

According to its major aspects and broadly stated, the present invention in its preferred form is a vocal pitch-training device having auditory, visual and physical biofeedback means.

More specifically, the present invention is a novel vocal pitch-training device that trains the user's mind to harmonize the function of the singer's vocal chords, mouth, throat, ears and diaphragm to reproduce and sustain a specified pitch at a selected note and/or key. Specifically, the device provides instantaneous auditory, visual and physical biofeedback that permits a user of the device to develop muscle and vocal memory to autonomously generate a specifically chosen “target note” or “target pitch” by teaching the user's mind to recognize complimentary and/or discordant auditory, visual, and physical vibrational signatures in the ears, throat, and body that inherently result from the user's conscious efforts to successfully match a vocally-generated “user-note” with a chosen “target note.”

The device involves the user's sense of sight, sound and touch—a triple-sense-biofeedback loop. Through use of the device, the user will learn to sing “on key” with proper pitch control by comparing the auditory, visual and physical biofeedback outputs from the device with the user's own vocal output delivered to the device as inputs, and thereafter, by user-modification of his/her own vocally-generated note or tone, to match his/her pitch with the auditory, visual and physical biofeedback outputs from the device as reference points. From such reference points, the user is able to instantaneously and intuitively adjust the frequency of his/her vocal output by elevating or lowering same until a desired harmony is achieved.

In one embodiment, the device is activated by a power-function key, wherein the user selects a “target note” (i.e., on a standard or programmable 12-note musical scale) and then depresses a corresponding note-function key. The device then displays the target note on an LCD screen; thus, providing the user with visual biofeedback. The device transmits the target note to the user via a tone generator worn in the user's ear (such as a headphone or e a r jack); thus, providing the user with auditory biofeedback. The device also transmits a vibration equal in tone or pitch to the target note to the user via a tone-generator or any other input vibrationally-responsive device worn against the user's throat, wrist, chest, head, or the like; thus, providing the user with tactile or physical biofeedback.

The user then attempts to generate a “user note” equal in pitch to the target note for input into the device via a microphone or similar device, whereby the device then analyzes the user-note and assigns a pitch or note value to the user note and displays it on the LCD screen; thus, further providing the user with visual biofeedback. The device continuously compares the pitch of the user-note to the pitch of the target note, and displays the correlation utilizing a progressive change in color of lights (i.e., visual feedback) that indicate to the user that the user's note or pitch is either flat, on-key or sharp, so that the user may adjust his/her voice output in an attempt to match the target note. Furthermore, if the user is off-key or off-pitch, pressure and/or discordant vibrations centered on the throat or vocal chord area are immediately felt by the user as a result of the discordant pitch or frequency between the user-note and the target note, thus providing additional physical bio-feedback.

When the pitch of the user note matches the pitch of the target note, the user will be visually notified by an LED light being displayed as green (or any other color). Additionally, and most importantly, the vibrations in the user's ear, throat and/or body produced by the physical or vibrational outputs of the device will also seemingly disappear, subside or become smooth in comparison to the vibrations of the vocal chords and ear drum of the user. Thus, complete auditory, visual and physical biofeedback is provided to the user.

It is contemplated that the present invention can be utilized to assist singer's reach of target notes, and/or could also be utilized to assist in physically rehabilitative speech efforts for the hearing impaired and/or those with injuries or physical impairments that have diminished their hearing and speech capacity, or by those seeking to adjust, modify, or improve their speaking voice in general.

Accordingly, a feature and advantage of the present invention is its incorporation of auditory, visual, and physical biofeedback mechanisms to provide a triple-biofeedback vocal-pitch training system.

Another feature and advantage of the present invention is its ability to provide expeditious results and instantaneous response via the novel triple-biofeedback system.

Still another feature and advantage of the present invention is its ability to harmonize the function of the singer's vocal chords, mouth, throat, ears and diaphragm to reproduce and sustain a specified pitch.

Yet another feature and advantage of the present invention is its ability to be utilized by any individual seeking to improve or develop specific vocal skills, wherein such individuals may include, but are not limited to, orators, singers, actors, academic students, and/or those who suffer from mental or physical disabilities that adversely impact vocal skills.

A further feature and advantage of the present invention is its ability to provide multiple biofeedback mechanisms that enable an individual lacking a key human sense (i.e., sight or touch) to utilize the device and all applicable and perceptible biofeedback mechanisms.

These and other features and advantages of the present invention will become more apparent to one skilled in the art from the following description and claims when read in light of the accompanying drawings.

### BRIEF DESCRIPTION OF THE DRAWINGS

The present invention will be better understood by reading the Detailed Description of the Preferred and Alternate Embodiments with reference to the accompanying drawing figures, in which like reference numerals denote similar structure and refer to like elements throughout, and in which:

FIG. 1 is a perspective view of a vocal pitch-training device according to a preferred embodiment of the present invention;

FIG. 2 is a perspective view of a headset of a vocal pitch-training device according to a preferred embodiment of the present invention; and,

FIG. 3 is a perspective view of an earpiece of a vocal pitch-training device according to an alternate embodiment of the present invention.

### DETAILED DESCRIPTION OF THE PREFERRED AND ALTERNATIVE EMBODIMENTS

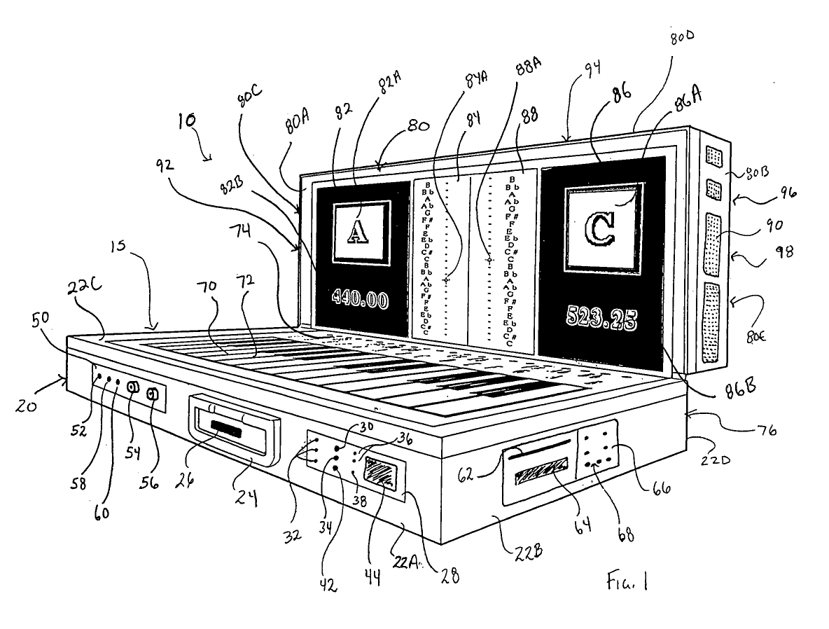

In describing the preferred and alternate embodiments of the present invention, as illustrated in FIGS. 1-3, specific terminology is employed for the sake of clarity. The invention, however, is not intended to be limited to the specific terminology so selected, and it is to be understood that each specific element includes all technical equivalents that operate in a similar manner to accomplish similar functions.

With regard to established sound wave theories, when two or more sources of sound produce notes whose frequencies are not in harmony with one another, a phenomenon known as “discordance” exists. The higher of the two frequencies cancels out the lower frequency and the resulting difference between them is transferred into the medium (i.e., air, water, or solids) in the form of a detectable vibration. When the human brain senses such vibrations within the body itself (including within the vocal chords or ear canal), it may interpret those insulting or offensive vibrations as “pain” or “discomfort.” It is a natural and normal instinct for a person to then employ some sort of strategy and course of action designed to avoid and/or terminate such an insulting or offensive vibration. Thus, if the insulting or offensive vibration is determined to be the result of a conflict between incoming sounds (i.e., background noises, conversations of others, and/or music) and outgoing vocalizations, then the natural and unconscious inclination of the person will be to alter their own outgoing vocalizations (pitch) until “harmony” between the incoming and outgoing tones is reached and the vibrations caused by discordance cease. Thus, the speaker/singer will bend the frequency of their own vocalizations either upward or downward slightly until “harmony” is achieved and the insulting or offensive vibrations lessen or are eliminated.

As it relates to the task of speaking or singing specific target notes, there are two general categories of humans: 1) those with “perfect pitch” (i.e., the singer can accurately produce any note from memory alone); and, 2) those who do not have such innate skills (i.e., the vast majority of humanity).

The difference between those two groups is “reference points.” Those with “perfect pitch” have internal references with which they can recall data, whereas most other singers need to hear at least one note produced through some type of sound generator (an instrument or another voice), wherein the note will then be utilized by the singer to guide him/her to vocalize in the proper key, and thus, enable the user to rely upon this/her training in proper musical (mathematical) intervals to stay on pitch while singing.

Accordingly, and as more fully described below, the present invention provides an electronic platform supporting separate levels of bio-feedback learning-loops (i.e., visual, auditory, and physical) which are designed to allow the user to experience, interact with, and learn (memorize) the look, sound, and feel of each individually selected target notes. As a result of the above process, the present invention enables the user to store more information concerning each target note during a practice session than is currently being experienced and memorized by utilizing one's ears alone (i.e., essentially a single, auditory-feedback loop); thus, providing the student with greater storage, recall, and reproduction of practiced notes when the student is engaged in the ultimate act of speaking or singing.

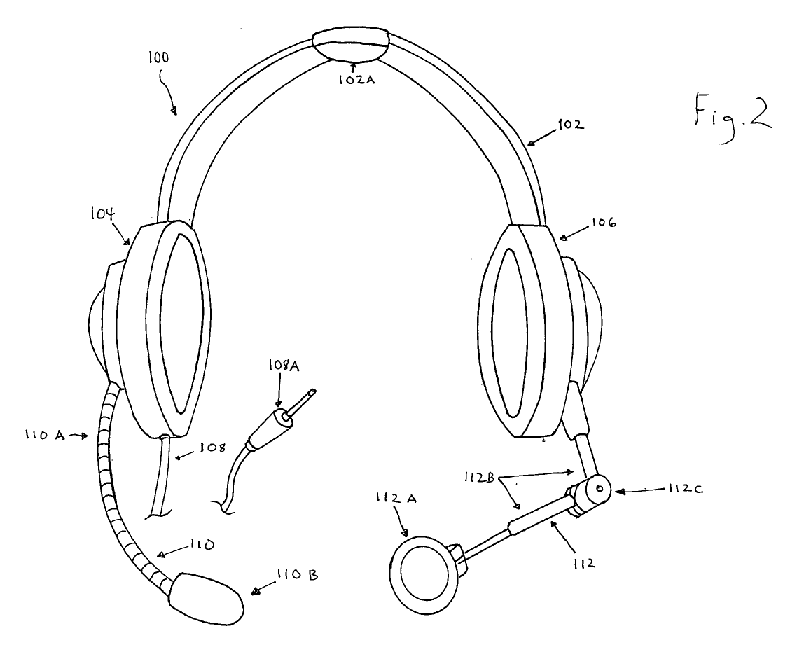

Referring now to FIGS. 1-2, the present invention, in a preferred embodiment, is a vocal pitch-training device 10, wherein device 10 preferably provides interactive main unit 15 and headset 100, wherein interactive main unit 15 and headset 100 preferably cooperatively function to provide a user of device 10 with the requisite auditory, visual and physical biofeedback responses necessary to effectively assist the user in vocal pitch training, improvement and/or development, as more fully described below.

Interactive main unit 15 is preferably provided in the form of a foldable case comprising console 20 hingably connected to visual display portion 80 and communicatively coupled thereto via suitable data transfer means known within the art, such as, for exemplary purposes only, ribbon or other cable, infrared ports, or the like. Preferably, console 20 and visual display portion 80, as well as headset 100, include the requisite interactive circuitry and electronics for operation of device 10, as known within the art. Additionally, console 20, and device 10 in general, preferably provide computer means for comparison and analysis of user generated notes and target notes for generating the requisite input or output signals for implementation of the preferred method, as more fully described below. Preferably, the downward folding of visual display portion 80 toward console 20 permits the convenient, compact storage and/or transport of device 10, wherein front side 22A of console 20 preferably provides centrally disposed handle 24 to facilitate transport or carriage of device 10 to a desired location.

Preferably disposed proximate to handle 24 is infrared port 26, wherein infrared port 26 preferably functions as a wireless data transmission means for device 10, transmitting and receiving desired information via infrared signals, as known within the art, and as more fully described below.

Preferably flanking infrared port 26 is first interface 28 and second interface 50, utilized to permit user interaction with, and system operation of, console 20 and visual display portion 80. Specifically, interface 28 preferably provides power key 30 for purposes of activating and deactivating device 10; octave range selection keys 32 for purposes of selecting the appropriate octave scale or range for a user of device 10; infrared power key 34 for purposes of activating and deactivating operation of infrared port 26; sector power keys 36 for purposes of selectively activating or deactivating sectors of visual display portion, as more fully described below; microphone power key 38 for purposes of selectively activating or deactivating a microphone means, as more fully described below; speaker power key 42 for selectively activating an external four-way or two-way speaker system for purposes as more fully described below; and digital display 44 for purposes of indicating systems operation of device 10, selected octave range of device 10, status of infrared transmission of infrared port 26, current status of activated or deactivated sectors of visual display portion 80, current status of the microphone means, and current status of speaker system. It is contemplated in an alternate embodiment that first interface 28 could possess any number and/or type of power-control or power-function keys for purposes of facilitating user-selected activation, deactivation, manipulation and/or adjustment of the various elements and components of device 10 as herein described.

Interface 50 preferably provides bi-directional socket 52 for the receipt and communicative coupling of headset 100 thereto, wherein bi-directional socket 52 preferably provides for the bi-directional transmission of audible and physical signals as generated by device 10 and by the user of device 10 during training sessions, as more fully described below. Interface 50 further preferably possesses volume control 54 for purposes of regulating the intensity of volume of the audible target signal generated by device 10 as conveyed through headset 100, as more fully described below; physical vibrational control 56 for purposes of regulating the intensity of the physical vibration generated by device 10 as conveyed through headset 100, wherein the physical vibration is preferably a direct correlative function of the pitch of the audible target signal generated by device 10, as more fully described below; and, auxiliary sockets 58 and 60 for purposes of outputting auditory and visual signals, respectively, for amplification by external auditory receiver sources and visual receiver sources, respectively, such as, for exemplary purposes only, surround-sound speaker systems and visual projectors, respectively. It is contemplated in an alternate embodiment that second interface 30 could possess any number and/or type of input or output sockets, and/or volume or signal intensity controls for purposes of facilitating user-selected manipulation and/or adjustment of the various auditory, visual and physical signals of device 10 as herein described.

Preferably, side 22B of console 20 provides CD-ROM drive 62, data disk drive 64, and associated power buttons 68 on interface 66, wherein CD-ROM drive 62 and data disk drive 64 preferably function to save, record, transmit, and/or install data and/or programs onto the computer circuitry of device 10, or onto compact disks or other data disks, as known within the art. CD-ROM drive 62 and data disk drive 64 are further preferably utilized to install specialized or upgraded vocal training programs or lessons onto device 10, and further function to record the training session and/or performance of a user of device 10 for subsequent analysis, critique and improvement of same.

Preferably, top surface 22C of console 20 provides keyboard 70 comprising bubble-type note keys 72, wherein keyboard 70 and note keys 72 are preferably based upon a conventional or standard programmable 12-note musical scale, and wherein note keys 72 are preferably electrically coupled to the electrical circuitry of console 20, and device 10 in general, as is known within the art. Preferably, indicator lights 74 corresponding to each individual note key 72, and positioned on top surface 20C of console 20, proximal to note keys 72, are activated upon depressing a specific note key 72, wherein activation of an indicator light 74 preferably results in activation of a corresponding light on visual display portion 80, delineating the selected note key 72, for purpose as more fully described below. Preferably, indicator lights 74 are in the form of light emitting diodes (LED), and preferably emit a green color when activated.

Preferably, back side 22D of console 20 provides a plurality of input and output sockets 76 for interactive coupling of any desired visual and auditory displays, or additional physical or tactile biofeedback devices, as more fully described below.

Preferably, front side 80A of visual display portion 80 comprises first graphical display interface 82 preferably positioned adjacent to target note reference display 84, and second graphical display interface 86 positioned adjacent to user note reference display 88, wherein target note reference display 84 and user note reference display 88 are preferably positioned adjacent one another, and thus, preferably separate first graphical display interface 82 and second graphical display interface 86 from one another, as best illustrated in FIG. 1. First graphical display interface 82 and second graphical display interface 86 are preferably liquid crystal displays (LCD); however, alternate forms of graphical displays could be utilized, such as, for exemplary purposes only, plasma screens, projectors, television screens, projection screens, or the like. It should be further recognized that alternate, equally effective, configurations or arrangements of visual display portion 80 could be utilized without departing from the appreciative scope of the present invention, as such additions and/or modifications to configuration or arrangement are in full contemplation of the inventor in describing the present invention herein. Additionally, visual display portion 80 could possess any number and type of graphical display interfaces to facilitate use of device 10.

As more fully detailed below, upon selection of a desired target note, and depressing the corresponding note key 72, a visually displayed target note 82A, as shown in Roman alphabet format, appears on first graphical display interface 82, and is further preferably indicated on target note reference display 84 via a corresponding target note indicator light 84A. A visually displayed frequency 82B corresponding to the frequency of the user-selected target note is further displayed on first graphical display interface 82, preferably in MHz/s. As indicated above, a corresponding indicator light 74 on console 20 is also preferably illuminated upon depressing a selected note key 72. Preferably, target note indicator lights 84A of target note reference display 84 are in the form of color changing light emitting diodes (LED), for purposes more fully described below.

Similarly, and also as more fully detailed below, upon generation of a vocal pitch by a user of device 10 in attempts to match the selected target note, and upon detection, analysis, and comparison of same by device 10, the user's generated note is translated into a visually displayed user note 86A on second graphical display interface 86, as shown in Roman alphabet format. The user's generated note is further preferably indicated on user note reference display 88 via a corresponding user note indicator light 88A, wherein a visually displayed frequency 86B corresponding to the frequency of the user's generated note is further displayed on second graphical display interface 86, preferably in MHz/s. Preferably, user note indicator lights 88A of user note reference display 88 are in the form of differently colored light emitting diodes (LED), for purposes more fully described below.

Preferably, sides 80B and 80C of visual display portion 80 possesses four-way external speaker systems 90 and 92, respectively, wherein speaker systems 90 and 92 are preferably activated via speaker power key 42 on first interface 28, as described above. Speaker systems 90 and 92 are preferably utilized when user of device 10 has garnered sufficient vocal skill so as to eliminate the need for the physical and auditory biofeedback supplied by headset 100, wherein activation of speaker systems 90 and 92 permit generation of the target note therethrough, as well as the looping of user's generated vocal note back into the system for analysis, comparison, and subsequent audible display through speaker systems 90 and 92, and visual display of the results (i.e., the user's note and frequency) on second graphical display interface 86 and user note reference display 88. When speaker systems 90 and 92 are in use, user of device 10 preferably transmits his/her vocally generated pitch to device 10 via a conventional microphone, wherein the microphone is preferably interactively coupled to device 10 via microphone socket 94 positioned on top side 80D of visual display portion 80.

Preferably, rear side 80E of visual display portion 80 possesses a compartmentalized storage housing 96 having hinged cover 98 thereover, wherein storage housing 96 is preferably adapted to receive and store spare LEDs, CD-ROMS, data disks, and headset 100 when in a folded state, as more fully described below.

Referring now to FIG. 2, headset 100 preferably comprises, head strap 102, earpieces 104 and 106, cable 108, microphone 110 and physical biofeedback mechanism 112, wherein headset 100 is preferably adjustably adapted to fit the head of any user, and wherein headset 100 is preferably collapsible or foldable via swivel or pivoting hinge 102A positioned on head strap 102.

Preferably, upon depressing a note key 72, the corresponding target note is audibly generated and conveyed to earpieces 104 and 106 via cable 108 for audible reception by the wearer of headset 100. Additionally, upon generation of a vocal pitch by a user of device 10 in attempts to match the selected audible target note being transmitted through earpieces 104 and 106, the user's generated note is conveyed through microphone 110, and through cable 108 for detection, analysis, and comparison of same by device 10, wherein the user's generated vocal pitch is thereafter re-conveyed or retransmitted back to headset 100 through earpieces 104 and 106 via cable 108 for auditory biofeedback purposes, as more fully developed below. Preferably microphone 110 possesses flexibly adjustable arm 110A and microphone head 110B, wherein arm 110A of microphone 110 is preferably attached to and extends from earpiece 104.

Preferably, upon depressing a note key 72, the corresponding target note is also translated into a physical vibration by device 10 and conveyed to physical biofeedback mechanism 112 via cable 108 for tactile or physical sensing or reception by the wearer of headset 100, wherein vibrational pad 112A is preferably placed against the wearer's throat, over the vocal cord region, for physical biofeedback purposes, as more fully described below. Preferably physical biofeedback mechanism 112 provides telescoping arm 112B having a pivoting mechanism 112C engaged therewith; thus, permitting user-adjustability of physical biofeedback mechanism 112 for purposes of more effectively positioning vibrational pad 112A against the user's throat. Telescoping arm 112B is preferably attached to and extends from earpiece 106; however, it is contemplated in an alternate embodiment that physical biofeedback mechanism 112 and microphone 110, in general, could be affixed to earpieces 104 and 106, respectively, or could alternatively, be removably interchangeable therebetween to accommodate the user thereof. Additionally, although headset 100 is preferred as a matter of convenience for compact functionality, it is contemplated in another alternate embodiment, that physical biofeedback mechanism 112 and microphone 110 could be integrated into the system in any other suitable fashion, such as, for exemplary purposes, separate cables and/or devices. It should be recognized that the term “physical” of physical biofeedback mechanism 112 is synonymous with the term “tactile,” wherein both terms are intended to semantically convey the idea that biofeedback mechanism 112 interacts with the user via the sense of touch and via vibrational sensory means.

Although physical biofeedback mechanism 112 is preferably in the form of a vibrational pad 112A placed against the throat of a wearer of headset 100, it is contemplated in an alternate embodiment that physical biofeedback mechanism 112 could be any other suitable mechanism capable of being worn or placed in contact with a user of device 10, and capable of delivering the requisite physical vibrations thereto, wherein such a mechanism could be in the form of, but not limited to, a helmet, shoe, wristband, belt, vest, nosepiece, earpiece, body suit, eyewear, skullcap, hat, headgear, forearm unit, gel floor mat, and/or combinations thereof. It is contemplated in an additional alternate embodiment that headset 100 could incorporate an earpiece that transmits both auditory and physical signals to the wearer. It is contemplated in yet another alternate embodiment that physical biofeedback mechanism 112, and/or headset 100 in general, could be wireless, operating via infrared signals with infrared port 26, or through other suitable wireless technologies.

Cable 108 of headset 100 is preferably interactively coupled to device 10 via inserting cable jack 108A of cable 108 into bi-directional socket 52 of interface 50 of console 20, wherein bi-directional socket 52 preferably provides for the bi-directional transmission of audible and physical signals as generated by device 10 and by the user of device 10 (i.e., wearer of headset 100) during training sessions, or the like. As indicated above, the intensity of volume of the audible target note signal generated by device 10 and conveyed through headset 100 is preferably controlled or regulated via volume control 54 of interface 50. Furthermore, the intensity of the physical vibration generated by device 10 and conveyed through vibrational pad 112 of headset 100 is preferably controlled or regulated via physical vibrational control 56 of interface 50, wherein the physical vibration is preferably a direct correlative function of the pitch of the audible target note signal generated by device 10.

In use, device 10 is preferably activated by depressing power key 30, wherein the user then preferably selects a “target note” (i.e., a target pitch or note that the user of device 10 wishes to vocally reproduce and practice) and depresses the corresponding note key 72 on keyboard 70. Preferably, upon depressing a specific note key 72, indicator light 74 on keyboard 70 illuminates; thus, resulting in illumination of corresponding target note indicator light 84A on target note reference display 84. Preferably simultaneously, visually displayed target note 82A appears on first graphical display interface 82, along with frequency 82B of the target note. Preferably, the target note indicator light 84A representing the selected target note is blue in color upon initial illumination and depression of the selected note key 72, as is the corresponding visually displayed target note 82A, wherein user generation of a vocal pitch in accord or harmony with the pitch of the selected target note preferably results in target note indicator light 84A and visually displayed target note 82A illuminating the color green, as more fully described below. As indicated above, the plurality of visual lights and displays as illustrated herein, and specifically with reference to visual display portion 80, preferably provide the user of device 10 with continuous visual biofeedback.

Furthermore, upon depression of selected note key 72, device 10 audibly transmits the target note or pitch to the user via earpieces 104 and 106 of headset 100; thus, supplying the user with auditory biofeedback. Device 10 further transmits a vibration equal in tone or pitch to the target note to the user via vibrational pad 112A of physical biofeedback mechanism 112; thus, providing the user with physical biofeedback, wherein vibrations equal to the pitch/frequency of the target note resonate against the user's throat area, and provide physical biofeedback to the user.

The user then attempts to vocally generate a “user note” or pitch equal in pitch to the target note, wherein the user's vocally generated user note is preferably conveyed to device 10 via microphone 110 of headset 100, whereby vibrations equal to the pitch or frequency of the vocally-generated user note are also preferably immediately felt by the user in the user's throat or vocal chord area via vibrational pad 112A, thus providing the user with physical biofeedback.

Device 10 then analyzes the user note, assigning a pitch or note value to the user note and displaying it on second graphical display interface 86 as visually displayed user note 86A; thus, providing the user with further visual biofeedback. The note or pitch of the user note is further preferably indicated on user note reference display 88 via a corresponding user note indicator light 88A, wherein a visually displayed frequency 86B corresponding to the frequency of the user's generated note is further displayed on second graphical display interface 86. Each indicator light 88A preferably corresponds to an individual musical note within a musical scale. Device 10 continuously compares the pitch of the user note to the pitch of the target note, and preferably displays the correlation or comparison via a progressive change in color of indicator lights 88A on user note reference display 88.

Specifically, upon initial generation of the user note, and analysis of same by device 10, the indicator light 88A corresponding to the pitch of the user note remains red in color for so long as the pitch of the user note differs from the pitch of the target note. As the user fluctuates or changes his/her vocal pitch in attempts to match the pitch of the target note audibly and physically sensed by the user via headset 100, and, thus, vocally moves up or down the musical scale as represented by indicator lights 88A, the indicator light 88A corresponding to each change in pitch or note begins to illuminate. When the user is able to generate a vocal pitch that corresponds to the indicator light 88A positioned three intervals or pitches away from the indicator light 88A that represents the pitch of the target note, the former indicator light 88A changes to yellow. When user is able to generate a vocal pitch that corresponds to the pitch of the target note, the indicator light 88A that represents the pitch of the target note becomes illuminated, preferably emitting a green light to indicate a perfect pitch match. The color of visually displayed user note 86A will also change or fluctuate in accord with changes in pitch of the user's generated note, remaining a single color only when the user's pitch is constant. Such a progressive change in color of indicator lights 88A and visually displayed user note 86A preferably visually notifies the user that the his/her note or pitch is either flat, on-key or sharp, so that the user may adjust his/her voice output in an attempt to match the target note; thus, providing the user with additional visual biofeedback.

Furthermore, if the user is off-key or off-pitch, the user will feel, via physical biofeedback form vibrational pad 112A, the pressure and/or discordant vibrations centered on the user's throat or voice box. The user's mind and body analyzes the user note in comparison with the auditory, visual and physical biofeedback from device 10 to the user, wherein if the user note is either “off-pitch” or “off-key”, the user will be immediately receptive of the discordant vocal performance due to the feeling of pressure and/or discordant vibrations centered on the user's throat and vocal chords. As such, when the pitch or frequency of the vocally-generated user note does not precisely match the pitch or frequency of the selected target note or any harmoniously correct alternative note in relation to the target note, the user must then attempt to correct his/her vocal output so as to be either “on-pitch” (i.e., the target note and user note are identical) or “on-key” (i.e., the user note represents a harmoniously correct alternative note in relation to the target note); thereby, reducing or negating the physical vibrations from vibrational pad 112A felt against the user's throat.

More specifically, when the pitch or frequency of the user note matches the pitch or frequency of the selected target note, the user will be visually notified by visually displayed target note 82A, target note indicator light 84A, visually displayed user note 86A, and user note indicator light 88A being displayed as green; and physically notified by the seeming dissipation of vibrations against the user's throat, wherein production of a user note that is either on-key or on-pitch with the target note will cause the sensation of physical vibrational outputs of vibrational pad 112A to seemingly disappear, subside or become smooth in comparison to the vibrations from the vocal chords (and ear canal of the user if serving as an auditory and physical biofeedback mechanism, as described above). In this manner, device 10 provides the user with the complete or final auditory, visual and physical biofeedback to train the user to sustain and reproduce target notes.

Device 10 will continue to display, generate and transmit the target note until the same note key 72 is depressed again or a different note key 72 selected. Device 10 will further continuously analyze and display the user note until the user ceases to speak or sing.

Preferably, it is contemplated that use of device 10 for vocal training purposes serves to strengthen the muscles of the throat, tongue, palette, jaw, and vocal chords for improved muscular flexibility and/or manipulation of same; thereby, reducing injury due to otherwise over-constrained or untrained muscles. Additionally, use of device 10 preferably functions to further strengthen the muscles of the diaphragm and chest wall; thus, allowing deeper lung inhalation and exhalation, and greater breath volume which translates to a stronger, steadier, more comfortable and accurate tonal projection of the user's voice.

Furthermore, preferably via visual notification by visually displayed user note 86A and user note indicator light 88A, and physical vibration via physical biofeedback mechanism 112, a user of device 10 is able to establish an immediate reference point upon initial vocal output, thus guiding the user to effectively achieve or reach the target note.

It is contemplated that device 10 could be utilized to assist in physically rehabilitative speech therapy for those suffering from either congenital speech impediments, partial or total deafness, or injury resulting from stroke or physical trauma to the throat, or related bodily areas that directly or indirectly influence vocal projection, enunciation and speech.

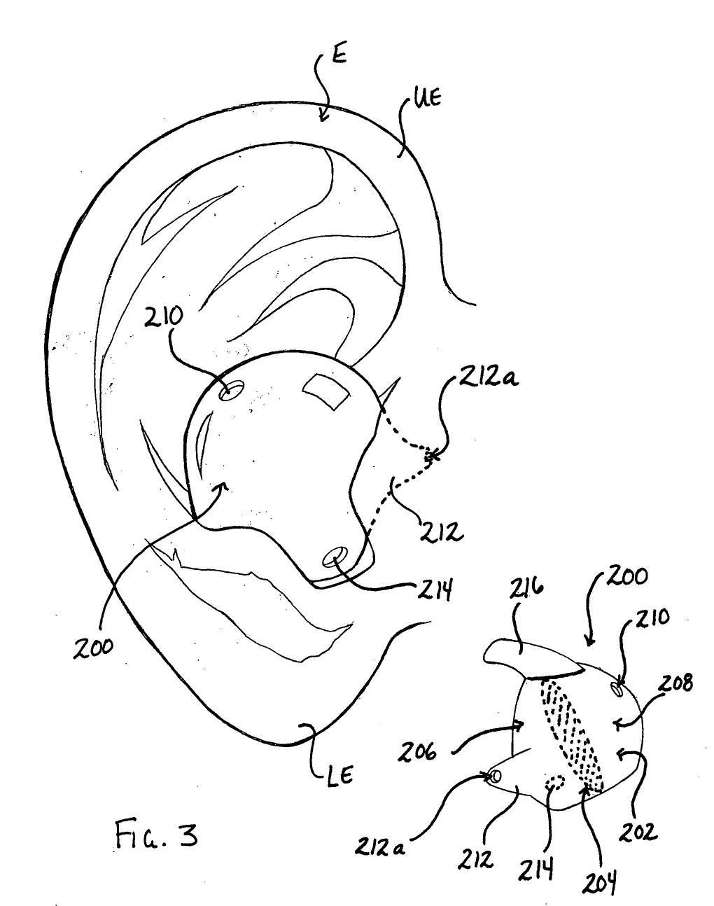

Referring now more specifically to FIG. 3, illustrated therein is an earpiece 200 for application in a method of vocal pitch-training according to an alternate embodiment of the present invention. Specifically, earpiece 200 is shaped and configured to be comfortably received within the user's ear E, and comprises inner chamber 202 bifurcated via vibratory membrane 204, thereby dividing inner chamber 202 into user-pitch chamber 206 and reference pitch chamber 208. Formed through earpiece 200, and in communication with reference pitch chamber 208, is aperture 210, through which an ambient audible reference note/pitch is permitted to enter, deflect against vibratory membrane 204, and exit back through aperture 210, thereby resulting in vibration of vibratory membrane 204, and providing the user with physical biofeedback due to the vibrations felt within the user's ear canal, as more fully described below.

Extending from earpiece 200, and in communication with user-pitch chamber 206, is frusto-conical-shaped protrusion 212 comprising aperture 212a formed therethrough and residing in communication with user-pitch chamber 206. Additionally, formed through earpiece 200, and also in communication with user-pitch chamber 206, is aperture 214. Earpiece 200 is worn in a user's ear E such that protrusion 212 is received within the user's ear canal, and such that aperture 214 is exposed and positioned closest to the user's mouth, proximal the user's lower earlobe LE. In such an orientation, aperture 210 is exposed and resides proximate to the user's upper earlobe UE. To assist in maintaining earpiece 200 within a user's ear E, fin 216 extends from earpiece 200, and is suitably dimensioned to be received within the folds of upper earlobe UE. Accordingly, and pursuant to well-established principles of sound wave travel, it should be recognized that vocalized user pitches or notes leave the user's mouth and travel in multiple directions—most notably, around the user's head. As such, upon generation of a user note/pitch, and subsequent travel of same around the user's head and toward the user's ear E, sound waves enter aperture 214, travel through user-pitch chamber 206, and strike vibratory membrane 204, thus resulting in additional vibration of same and providing the user with physical biofeedback due to corresponding vibrations felt within the user's ear canal. As the user senses the vibrationally discordant biofeedback between his/her vocalized pitch and the ambient reference pitch as a result of the discordantly vibrating vibratory membrane 204, the user adjusts his/her vocalized pitch in an attempt to minimize the discordant biofeedback as recognized by a seemingly corresponding diminishment of physical biofeedback (i.e., diminishment or elimination of sensation of vibration within the user's ear canal).

It should be recognized that earpiece 200 could further comprise the requisite nanotechnology adapted to wirelessly interact with interactive main unit 15.

It is contemplated in an alternate embodiment that device 10 may be configured in any arrangement conducive to transmitting auditory, physical and visual biofeedback to the user thereof.

It is contemplated in another alternate embodiment that a user of device 10 could utilize the auditory, physical and visual biofeedback mechanisms of the present invention either individually or in a selected combination thereof; thereby, effectuating a user-designed pitch training process.

It is contemplated in still another alternate embodiment that the vibrational intensity of physical biofeedback mechanism 112 could be automatically regulated by device 10, wherein the vibrational sensation felt by the user would correspondingly increase or decrease as the frequency of the user's pitch moved farther from or closer to the target pitch.

It is contemplated in yet another alternate embodiment that wall mountable flat screen visual displays or panels could also be utilized to effectuate visual biofeedback.

It is contemplated in still yet another alternate embodiment that software programs may be utilized in conjunction with device 10 to permit sheet music to stream across graphical interface displays 82 and 86.

It is contemplated in a further alternate embodiment that a karaoke machine, either adjunctively associated or integrally formed with device 10, may be utilized in combination therewith to permit “target” sheet music, audible music, and/or lyrics to stream across graphical interface display 82, wherein a user of the present alternate device would then attempt to vocally match the “target” sheet music, audible music, and/or lyrics for analysis and comparison by device 10 via an equivalent method as described herein, and wherein the results of the user's analyzed and compared vocally generated response would then be visual displayed on graphical interface display 86, physically sensed via physical biofeedback means 112 and audible sensed via headset 100, or via any other suitable alternate embodiment described herein or otherwise.

It is contemplated in still a further alternate embodiment that device 10 could be coupled to the Internet, or other global networking systems, for downloading training programs, effectuating live interactive online training sessions, and/or updating system operations of device 10.

It is contemplated in yet a further alternate embodiment that device 10 could entirely be in the form of a helmet.

It is contemplated in another and further alternate embodiment that device 10 could entirely be in the form of a handheld or wearable unit, ideally in the form of a handheld microphone capable of conveying and receiving audible biofeedback signals, visual biofeedback signals via a visual display mounted thereon or in association therewith, and physical biofeedback signals via the hand grip on the microphone, wherein all such biofeedback signals would be conveyed and received via bi-directional cabling and/or other suitable transmission mediums.

Having thus described exemplary embodiments of the present invention, it should be noted by those skilled in the art that the within disclosures are exemplary only, and that various other alternatives, adaptations, and modifications may be made within the scope of the present invention. Accordingly, the present invention is not limited to the specific embodiments illustrated herein, but is limited only by the following claims.

What is claimed is:

1. A vocal training device, comprising:
means for tactile biofeedback, said tactile biofeedback means adapted to assist a vocal trainee achieve a desired vocal output.

2. The vocal training device of claim 1, further comprising an interactive unit adapted to compare and analyze a vocal trainee generated note against a target note generated by said interactive unit.

3. The vocal training device of claim 2, wherein the vocal trainee generated note is conveyed to said interactive unit via a microphone.

4. The vocal training device of claim 3, wherein said target note is audibly generated by said interactive unit by selecting a corresponding target note key.

5. The vocal training device of claim 4, further comprising means for auditory biofeedback, said auditory biofeedback means adapted to assist the vocal trainee achieve a desired vocal output.

6. The vocal training device of claim 5, wherein said auditory biofeedback means is an earpiece.

7. The vocal training device of claim 6, wherein audibly generated said target note is conveyed to said earpiece for audible reception and biofeedback to the vocal trainee.

8. The vocal training device of claim 7, wherein the vocal trainee generated note is conveyed to said interactive unit, compared and analyzed against said target note, and subsequently looped back to said earpiece for audible reception and biofeedback to the vocal trainee.

9. The vocal training device of claim 8, further comprising means for visual biofeedback, said visual biofeedback means adapted to assist the vocal trainee achieve a desired vocal output.

10. The vocal training device of claim 9, wherein said visual biofeedback means is a visual graphical interface for visually conveying vocal training information to the vocal trainee.

11. The vocal training device of claim 10, wherein said target note is visually generated on said visual graphical interface by said interactive unit by selecting a corresponding target note key.

12. The vocal training device of claim 11, wherein visually generated said target note is in Roman alphabet format corresponding to said target note.

13. The vocal training device of claim 12, wherein visually generated said target note is in the form of an indicator light corresponding to said target note.

14. The vocal training device of claim 13, wherein the vocal trainee generated note is conveyed to said interactive unit, compared and analyzed against said target note, and subsequently displayed in Roman alphabet format on said visual graphical interface for visual comparison of same against said target note also displayed in Roman alphabet format on said visual graphical interface.

15. The vocal training device of claim 14, wherein the vocal trainee generated note is conveyed to said interactive unit, compared and analyzed against said target note, and subsequently displayed as an indicator light on said visual graphical interface for visual comparison of same against said target note also displayed as an indicator light on said visual graphical interface.

16. The vocal training device of claim 3, wherein said tactile biofeedback means is a physical vibration sensed by the vocal trainee.

17. The vocal training device of claim 16, wherein said tactile biofeedback means is selected from the group consisting of vibrational pads worn against the throat of the vocal trainee, vibrational helmets, vibrational earpiece, vibrational nosepiece, vibrational shoes, vibrational wristband, vibrational vest, vibrational chest piece, vibrational belt, vibrational body suit, vibrational eyewear, vibrational skullcap, vibrational head apparel, vibrational headgear, vibrational forearm unit, vibrational gel floor mat, and combinations thereof.

18. The vocal training device of claim 17, wherein said target note is translated into a physical vibration by said interactive unit by selecting a corresponding target note key, and wherein said physical vibration is subsequently conveyed to the vocal trainee for physical or tactile perception of same.

19. The vocal training device of claim 18, wherein adjusting the vocal trainee generated note to match said target note, and thus minimize discordance between same, results in a seemingly corresponding diminishment of said physical vibration sensed by the vocal trainee.

20. A vocal training device, comprising:
means for tactile biofeedback;
means for auditory biofeedback; and,
means for visual biofeedback,
wherein said tactile biofeedback means, said auditory biofeedback means and said visual biofeedback means are adapted to assist a vocal trainee achieve a desired vocal output.

21. The vocal training device of claim 20, further comprising an interactive unit adapted to compare and analyze a vocal trainee generated note against a target note generated by said interactive unit.

22. The vocal training device of claim 21, wherein the vocal trainee generated note is conveyed to said interactive unit via a microphone.

23. The vocal training device of claim 22, wherein said target note is audibly generated by said interactive unit by selecting a corresponding target note key.

24. The vocal training device of claim 23, wherein said auditory biofeedback means is an earpiece.

25. The vocal training device of claim 24, wherein audibly generated said target note is conveyed to said earpiece for audible reception and biofeedback to the vocal trainee.

26. The vocal training device of claim 25, wherein the vocal trainee generated note is conveyed to said interactive unit, compared and analyzed against said target note, and subsequently looped back to said earpiece for audible reception and biofeedback to the vocal trainee.

27. The vocal training device of claim 26, wherein said visual biofeedback means is a visual graphical interface for visually conveying vocal training information to the vocal trainee.

28. The vocal training device of claim 27, wherein said target note is further visually generated on said visual graphical interface by said interactive unit by selecting said corresponding target note key.

29. The vocal training device of claim 28, wherein the vocal trainee generated note is conveyed to said interactive unit, compared and analyzed against said target note, and subsequently displayed in Roman alphabet format on said visual graphical interface for visual comparison of same against said target note also displayed in Roman alphabet format on said visual graphical interface.

30. The vocal training device of claim 29, wherein the vocal trainee generated note is conveyed to said interactive unit, compared and analyzed against said target note, and subsequently displayed as an indicator light on said visual graphical interface for visual comparison of same against said target note also displayed as an indicator light on said visual graphical interface.

31. The vocal training device of claim 30, wherein said tactile biofeedback means is a physical vibration sensed by the vocal trainee.

32. The vocal training device of claim 31, wherein said tactile biofeedback means is selected from the group consisting of vibrational pads worn against the throat of the vocal trainee, vibrational helmets, vibrational earpiece, vibrational nosepiece, vibrational shoes, vibrational wristband, vibrational vest, vibrational chest piece, vibrational belt, vibrational body suit, vibrational eyewear, vibrational skullcap, vibrational head apparel, vibrational headgear, vibrational forearm unit, vibrational gel floor mat, and combinations thereof.

33. The vocal training device of claim 32, wherein said target note is translated into a physical vibration by said interactive unit by selecting said corresponding target note key, and wherein said physical vibration is subsequently conveyed to the vocal trainee for physical or tactile perception of same.

34. The vocal training device of claim 33, wherein adjusting the vocal trainee generated note to match said target note results in harmonization of said target note and the vocal trainee generated note as audibly perceived by the vocal trainee via said earpiece.

35. The vocal training device of claim 34, wherein adjusting the vocal trainee generated note to match said target note results in the vocal trainee generated note being reassigned a note value displayed in said Roman alphabet format corresponding to or matching said target note as displayed in said Roman alphabet format on said visual graphical interface.

36. The vocal training device of claim 35, wherein adjusting the vocal trainee generated note to match said target note results in the vocal trainee generated note being reassigned a note value displayed as said indicator light corresponding to or matching said target note as displayed as another said indicator light on said visual graphical interface.

37. The vocal training device of claim 35, wherein adjusting the vocal trainee generated note to match said target note results in a corresponding and progressive change in color of said indicator light to match a stagnate color of another said indicator light corresponding to said target note as displayed on said visual graphical interface.

38. The vocal training device of claim 35, wherein adjusting the vocal trainee generated note to match said target note results in a corresponding and progressive change in color of a series of indicator lights to match a stagnate color of an indicator light corresponding to said target note as displayed on said visual graphical interface.

39. The vocal training device of claim 36, wherein adjusting the vocal trainee generated note to match said target note, and thus minimize discordance between same, results in a seemingly corresponding diminishment of said physical vibration sensed by the vocal trainee.

40. The vocal training device of claim 39, further comprising an external speaker system for providing the vocal trainee with additional auditory biofeedback.

41. The vocal training device of claim 40, further comprising an means for recoding the vocal trainee's vocal training session for subsequent analysis of same.

42. A method of vocal training, comprising the steps of:
a. generating a vocal pitch; and,
b. adjusting the vocal pitch to match a target note translated into a sensed biofeedback, said sensed biofeedback selected from the group consisting of visual biofeedback, auditory biofeedback, tactile biofeedback, and combinations thereof.

43. A method of vocal training, comprising the steps of:
a. obtaining a vocal training device, comprising:
means for tactile biofeedback;
means for auditory biofeedback; and,
means for visual biofeedback; and,
b. selecting a target note for vocal reproduction;
c. generating an auditory pitch corresponding to the pitch of said target note;
d. audibly recognizing said auditory pitch via said auditory biofeedback means;
e. visually recognizing said auditory pitch via said visual biofeedback means;
f. generating a physical vibration corresponding to the frequency of said target note;
g. tactilely recognizing said physical vibration via said tactile biofeedback means;
h. producing said auditory pitch into a vocalized pitch;
i. sensing the discordant biofeedback between said auditory pitch and said vocalized pitch via said tactile biofeedback means; and,
j. adjusting said vocalized pitch to match said auditory pitch by minimizing said discordant biofeedback as recognized by a seemingly corresponding diminishment of said physical vibration.

44. A vocal training device, comprising:
an earpiece adapted to vibrate upon encounter of user-generated sound waves, thereby providing the user with tactile biofeedback.

45. The vocal training device of claim 44, further comprising a main chamber bifurcated by a vibratory membrane.

46. The vocal training device of claim 45, wherein a first chamber of said main chamber is adapted to receive and deflect an ambient reference pitch, thereby resulting in vibration of said vibratory membrane.

47. The vocal training device of claim 46, wherein a second chamber of said main chamber is adapted to receive the user-generated sound waves for disruption, and thus, further vibration of said vibratory membrane.

48. The vocal training device of claim 47, wherein vibrations from said vibratory membrane are channeled into the user's ear canal for tactile reception and sensing of same as tactile biofeedback.

49. The vocal training device of claim 48, wherein user sensation of discordant biofeedback between the user-generated sound waves and said ambient reference pitch, as sensed by the user via discordant vibrations delivered via said vibrating vibratory membrane, enables the user to adjust his/her user-generated sound waves in an attempt to minimize the discordant biofeedback as recognized by a seemingly corresponding diminishment of said tactile biofeedback.
